(19) United States
(12) Patent Application Publication
(10) Pub. No.: US 2016/0317081 A1
(43) Pub. Date: Nov. 3, 2016

(54) DISPOSABLE, IN-LINE ENTEROTOMY DETECTION DEVICE

(71) Applicant: THE METHODIST HOSPITAL, Houston, TX (US)

(72) Inventor: Albert Yung-Hsiang Huang, Houston, TX (US)

(21) Appl. No.: 15/108,124

(22) PCT Filed: Dec. 29, 2014
(86) PCT No.: PCT/US14/72574

§ 371 Date: Jun. 24, 2016

Related U.S. Application Data
(60) Provisional application No. 61/921,206, filed on Dec. 27, 2013.

(51) Int. Cl. A61B 5/00; A61B 17/34; A61M 39/10; A61M 13/00

(52) U.S. Cl. CPC A61B 5/4836; A61M 13/006; A61B 5/4255; A61B 17/3423; A61B 17/3474; A61M 39/10; G01N 21/783

(57) ABSTRACT

An in-line enterotomy detection device for use in a surgical procedure wherein access to an internal operating field is provided through a plurality of access ports and further wherein a pressurized insufflating gas is delivered to the internal operating field through one access port and vented from the internal operating field through another access port, the in-line enterotomy detection device comprising: a housing having an inlet, an outlet, and a passageway connecting the inlet to the outlet, the inlet being configured for connection to an access port venting gas from the internal operating field and the outlet being configured to vent gas from the in-line enterotomy detection device; and at least one non-electrical colorimetric enteric gas sensor disposed within the passageway for determining at least one of the presence of a selected enteric gas and the concentration of a selected enteric gas and indicating that presence by a visually-apparent change in appearance.

### REFERENCE TO PENDING PRIOR PATENT APPLICATION

This patent application claims benefit of pending prior U.S. Provisional Patent Application Ser. No. 61/921,206, filed Dec. 27, 2013 by The Methodist Hospital and Albert Yung-Hsiang Huang for DISPOSABLE, IN-LINE ENTEROTOMY DETECTION SYSTEM (Attorney's Docket No. METHODIST-7 PROV), which patent application is hereby incorporated herein by reference.

### FIELD OF THE INVENTION

This invention relates to medical apparatus and procedures in general, and more particularly to medical methods and apparatus for detecting enterotomies (i.e., perforations of the bowel).

### BACKGROUND OF THE INVENTION

With minimally invasive surgical procedures and robotic procedures becoming the accepted standard of care for many medical conditions, a number of risks associated with these procedures have become increasingly apparent. Among other things, accidental enterotomies (i.e., unintentional perforations of the bowel) are a well-known risk of such procedures, which can occur during initial intraabdominal access, port placements, tissue dissections, anatomy mobilization, etc.

More particularly, minimally invasive procedures and/or robotic procedures typically require that the surgeon rely on endoscopes and video monitors or other imaging technology (e.g., fluoroscopy, ultrasound, etc.) to provide a view of the internal operating field. This is, ultimately, a limited field of view, and processes occurring outside of this limited field of view may easily go undetected. As a result, unintended contact with the patient's tissue may occur. In the area of the gastrointestinal tract, such unintended contact with the patient's tissue may result in unintended perforations of the bowel (i.e., unplanned enterotomies). By way of example but not limitation, with cases such as bariatric procedures, where large amounts of fatty tissue are typically present in the surgical field, and/or where the patient may have undergone previous operations and hence may have extensive scarring, adhesions, etc., the internal operating field becomes even more complex and presents a higher degree of risk for unintended perforations.

An accidental (i.e., unplanned) enterotomy which is detected during a procedure can typically be repaired during the same procedure, but an undetected enterotomy can lead, post-operatively, to significant increases in patient morbidity and mortality, as well as increased hospital costs. Patients who suffer an undetected enterotomy often require intensive care, a corrective procedure and, in some cases, long-term follow-up care. In extreme cases, death may even result.

Thus there exists a need for a small, low-cost, non-electrical, disposable device for monitoring a patient during a minimally invasive procedure and/or robotic procedure (and/or other procedure), detecting the occurrence of an unplanned enterotomy during the procedure, and alerting the surgeon and operating room personnel to the occurrence of the unplanned enterotomy during the procedure, so as to allow for appropriate correction of the unplanned enterotomy during the procedure and thereby avoid subsequent postoperative complications.

### SUMMARY OF THE INVENTION

The present invention comprises the provision and use of a small, low-cost, non-electrical, disposable device for monitoring a patient during a minimally invasive procedure and/or robotic procedure (and/or other procedure), detecting the occurrence of an unplanned enterotomy during the procedure, and alerting the surgeon and operating room personnel to the occurrence of the unplanned enterotomy during the procedure, so as to allow for appropriate correction of the unplanned enterotomy during the procedure and thereby avoid subsequent postoperative complications.

More particularly, the present invention comprises the provision and use of a small, low-cost, non-electrical, disposable device that monitors the gas composition present in the intraabdominal cavity during minimally invasive procedures and/or robotic procedures (and/or other procedures) and senses for the presence of gases that are typically found only within the bowel and/or senses for the presence of gas concentrations that are typically found only within the bowel. By monitoring the gases within the intraabdominal cavity during minimally invasive procedures and/or robotic procedures (and/or other procedures), and detecting the presence of gases that are typically found only within the bowel and/or detecting the presence of gas concentrations that are typically found only within the bowel, the surgeon and operating room personnel can be alerted, during the minimally invasive procedure and/or robotic procedure (and/or other procedure), of the likelihood that an unplanned enterotomy has occurred. This then allows the surgeon to take appropriate corrective action during the procedure, whereby to avoid subsequent postoperative complications.

In one preferred form of the present invention, there is provided an in-line enterotomy detection device for use in a surgical procedure wherein access to an internal operating field is provided through a plurality of access ports and further wherein a pressurized insufflating gas is delivered to the internal operating field through one access port and vented from the internal operating field through another access port, said in-line enterotomy detection device comprising:

a housing having an inlet, an outlet, and a passageway connecting said inlet to said outlet, said inlet being configured for connection to an access port venting gas from the internal operating field and said outlet being configured to vent gas from said in-line enterotomy detection device; and

at least one non-electrical colorimetric enteric gas sensor disposed within said passageway for determining at least one of the presence of a selected enteric gas and the concentration of a selected enteric gas and indicating that presence by a visually-apparent change in appearance.

In another preferred form of the present invention, there is provided a novel method for detecting perforation of the bowel in a surgical procedure wherein access to an internal operating field is provided through a plurality of access ports and further wherein a pressurized insufflating gas is delivered to the internal operating field through one access port and vented from the internal operating field through another access port, said method comprising:

providing an in-line enterotomy detection device comprising:
a housing having an inlet, an outlet, and a passageway connecting said inlet to said outlet; and
at least one non-electrical colorimetric enteric gas sensor disposed within said passageway for determining at least one of the presence of a selected enteric gas and the concentration of a selected enteric gas and indicating that presence by a visually-apparent change in appearance;

connecting said inlet to an access port venting gas from the internal operating field; and

observing the appearance of said at least one non-electrical colorimetric enteric gas sensor during the surgical procedure.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the abdominal area of a patient during a typical minimally invasive and/or robotic procedure (and/or other procedure);

FIGS. 2 and 3 are schematic views showing a small, low cost, non-electrical, disposable device formed in accordance with the present invention;

FIGS. 4-7 are schematic views showing various aspects of the construction of the device shown in FIGS. 2 and 3; and

FIG. 8 is a schematic view showing another possible configuration for the present invention.

### DETAILED DESCRIPTION OF THE PREFERRED EMBODIMENTS

The present invention comprises the provision and use of a small, low-cost, non-electrical, disposable device for monitoring a patient during a minimally invasive procedure and/or robotic procedure (and/or other procedure), detecting the occurrence of an unplanned enterotomy during the procedure, and alerting the surgeon and operating room personnel to the occurrence of the unplanned enterotomy during the procedure, so as to allow for appropriate correction of the unplanned enterotomy during the procedure and thereby avoid subsequent postoperative complications.

More particularly, the present invention comprises the provision and use of a small, low-cost, non-electrical, disposable device that monitors the gas composition present in the intraabdominal cavity during minimally invasive procedures and/or robotic procedures (and/or other procedures) and senses for the presence of gases that are typically found only within the bowel and/or senses for the presence of gas concentrations that are typically found only within the bowel. By monitoring the gases within the intraabdominal cavity during minimally invasive procedures and/or robotic procedures (and/or other procedures), and detecting the presence of gases that are typically found only within the bowel and/or detecting the presence of gas concentrations that are typically found only within the bowel, the surgeon and operating room personnel can be alerted, during the minimally invasive procedure and/or robotic procedure (and/or other procedure), of the likelihood that an unplanned enterotomy has occurred. This then allows the surgeon to take appropriate corrective action during the procedure, whereby to avoid subsequent postoperative complications.

Various studies have identified the typical composition of enteric (i.e., intestinal) gas. Nitrogen, hydrogen, carbon dioxide and methane typically make up the majority of enteric gas. Hydrogen sulfide, dimethylsulfide and methanethiol (aka methyl mercaptan) are also typically present in enteric gas.

With minimally invasive procedures and/or robotic procedures (and/or other procedures), the abdomen is typically inflated with carbon dioxide so as to provide improved surgical access and visualization, so sensing the presence of carbon dioxide within the intraabdominal cavity is of little use in identifying the occurrence of an unplanned enterotomy.

Nitrogen is prevalent in ambient air, so sensing the presence of nitrogen within the intraabdominal cavity is also of little use in identifying the occurrence of an unplanned enterotomy.

However, hydrogen sulfide, dimethylsulfide and methanethiol (aka methyl mercaptan) are not commonly found outside the bowel, so sensing the presence of hydrogen sulfide, dimethylsulfide and/or methanethiol (aka methyl mercaptan) within the intraabdominal cavity can be an excellent and specific indicator of the occurrence of an unplanned enterotomy.

In addition, approximately ⅓ of patients produce substantial quantities of methane, so sensing a significant increase in the concentration of methane within the intraabdominal cavity can also be an excellent indicator of the occurrence of an unplanned enterotomy.

Furthermore, hydrogen gas is a known product of fermentation in the human intestinal tract and is found in all individuals beginning approximately 48 hours after birth. Therefore, sudden changes in the concentration of hydrogen gas in the intraabdominal cavity can also be an excellent indicator of an intestinal perforation.

The present invention provides a small, low-cost, non-electrical, disposable device for monitoring the gas present in the intraabdominal cavity during a minimally invasive procedure and/or a robotic procedure (and/or other procedure) and sensing for the presence of gases that are typically found only within the bowel (e.g., hydrogen sulfide, dimethylsulfide and methanethiol, aka methyl mercaptan) and/or sensing for the presence of gas concentrations that are typically found only within the bowel (e.g., high concentrations of methane, sudden changes in the concentration of hydrogen gas, etc.).

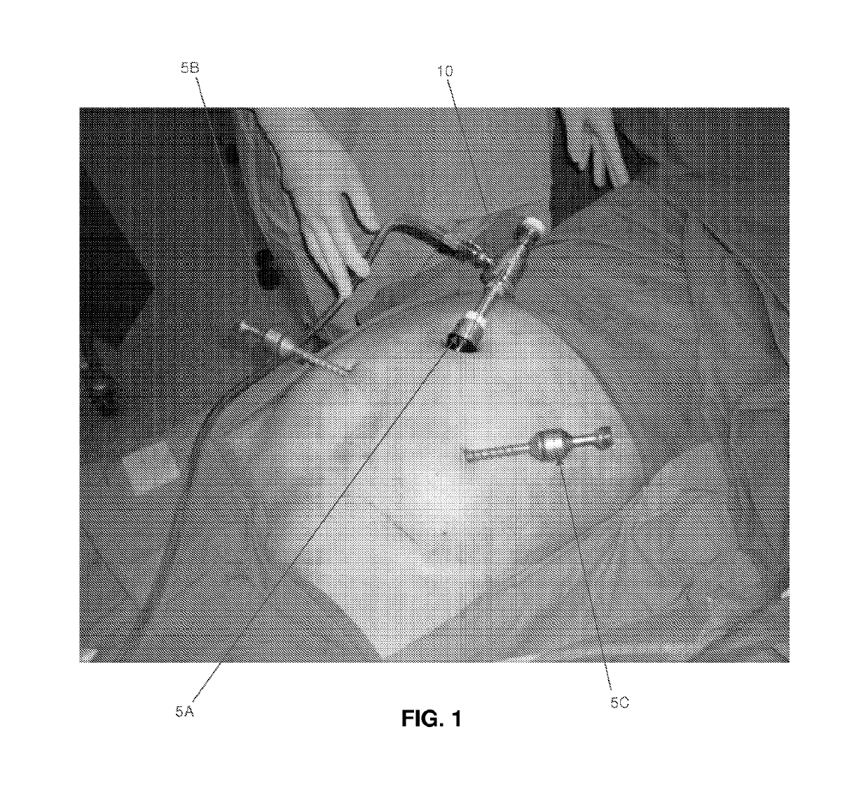

More particularly, FIG. 1 shows the abdominal area of a patient during a typical minimally invasive procedure and/or robotic procedure (and/or other procedure). In such a procedure, a plurality of access ports (sometimes referred to as access cannulas or access trocars) 5 are commonly used to provide a plurality of portals (sometimes referred to as surgical corridors or access passageways) between the skin of the patient and the intraabdominal cavity. Typically, one access port 5 (e.g., access port 5A) receives an endoscope 10 for allowing the surgeon to view the internal operating field. Other access ports 5 (e.g., access ports 5B and 5C) receive the surgical instruments used to perform the minimally invasive procedure and/or robotic procedure (and/or other procedure). Gas (e.g., carbon dioxide) is typically introduced through one of the access ports 5 (or through the endoscope 10) so as to allow inflation of the abdomen during the minimally invasive procedure and/or robotic procedure (and/or other procedure), whereby to provide better surgical access to the internal operating field and/or better visualization of the internal operating field.

In accordance with the present invention, a small, low-cost, non-electrical, disposable device is provided for monitoring the gas present in the intraabdominal cavity during a minimally invasive procedure and/or a robotic procedure (and/or other procedure) and sensing for the presence of gases that are typically found only within the bowel (e.g., hydrogen sulfide, dimethylsulfide and methanethiol, aka methyl mercaptan) and/or sensing for the presence of gas concentrations that are typically found only within the bowel (e.g., high concentrations of methane, sudden changes in the concentration of hydrogen gas, etc.), whereby to permit real-time detection of an unplanned enterotomy.

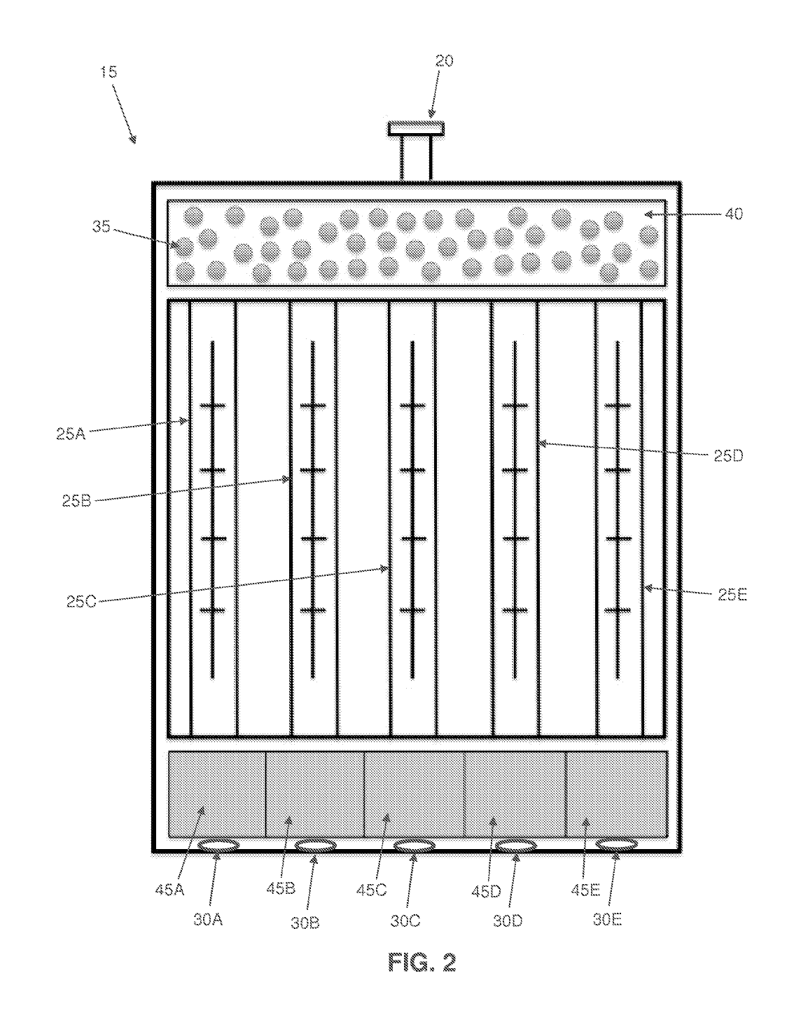

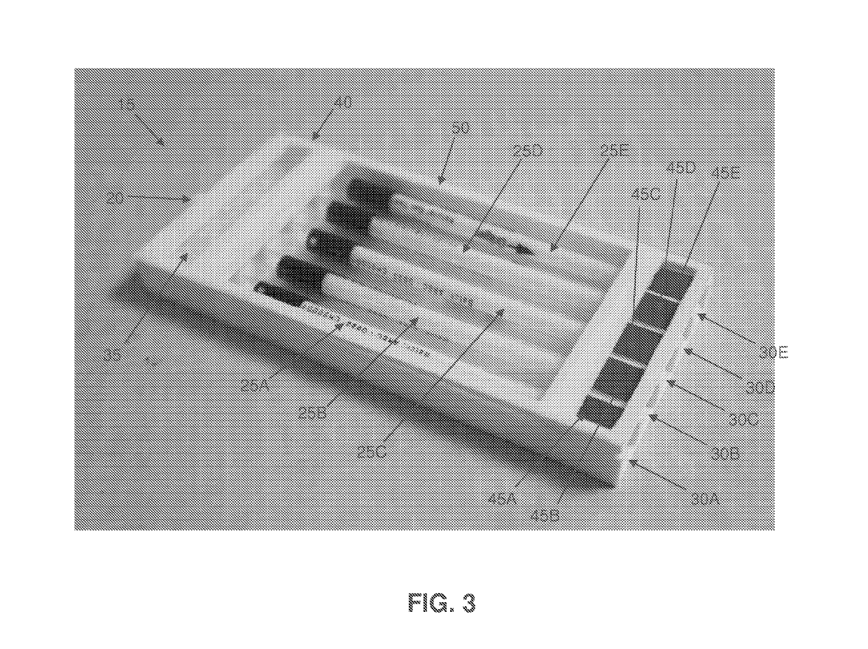

More particularly, and looking now at FIGS. 2 and 3, in one preferred form of the invention, there is provided a small, low-cost, non-electrical, disposable device 15. Device 15 is preferably approximately the size of a deck of playing cards. Device 15 generally comprises an inlet 20 for receiving intraabdominal air (e.g., from the side gas valve of an access port, e.g., access port 5B or 5C), a plurality of individual colorimetric enteric gas sensors 25A, 25B, 25C, 25D, 25E, etc. disposed within the interior of device 15 and communicating with inlet 20 for determining the presence of selected enteric gases and/or concentrations of selected enteric gases in the intraabdominal air admitted by inlet 20 and indicating that presence by a visually-apparent change in appearance, and a plurality of outlets 30A, 30B, 30C, 30D, 30E, etc. communicating with the individual colorimetric enteric gas sensors 25A, 25B, 25C, 25D, 25E, etc., respectively, for venting intraabdominal air from device 15 (e.g., into the ambient atmosphere, into another downstream device, etc.).

Thus, device 15 is configured to be disposed “in-line”, in the sense that it is intended to be connected intermediate an air vent pathway (e.g., between the side gas valve of an access port such as access port 5B or 5C and the ambient atmosphere or another downstream device).

Preferably device 15 is completely non-electrical, and is otherwise constructed, so that gas from the intraabdominal cavity may be fed directly to the individual colorimetric enteric gas sensors 25A, 25B, 25C, 25D, 25E, etc., without requiring the presence of any intervening filters (which add complexity and increase the risk of clogging).

Device 15 is constructed so that the plurality of individual colorimetric enteric gas sensors 25A, 25B, 25C, 25D, 25E, etc. are readily visible to the surgeon and operating room personnel. Preferably, each individual colorimetric enteric gas sensor 25A, 25B, 25C, 25D, 25E, etc. comprises a separate sensing tube for determining the presence of a particular enteric gas (e.g., by changing color) and/or the presence of a threshold concentration of a particular enteric gas and indicating that presence by a visually-apparent change in appearance (e.g., by a change in color when a threshold concentration is reached, or by the distance of color change down the length of each sensing tube to indicate gas concentration). If desired, where the intensity of the color change reflects the presence of a particular enteric gas and/or a concentration of a particular enteric gas, device 15 can have a printed color standard disposed on or accompanying the device in order to assist the user in interpreting the significance of the color change, e.g., such as where a light blue color indicates the presence of a particular enteric gas and a dark blue color indicates an overabundance of a particular enteric gas, etc.

Note that, if desired, more than one individual colorimetric enteric gas sensor 25A, 25B, 25C, 25D, 25E, etc. may be provided for determining and indicating the presence of a particular enteric gas (e.g., by changing color) and/or the presence of a threshold concentration of a particular enteric gas, i.e., device 15 can provide for redundancy.

Device 15 preferably also comprises a colorimetric humidity/fluid saturation sensor 35 for detecting if excessive fluid is present in the intraabdominal gas and, if excessive fluid is present in the intraabdominal gas, indicating the same by a visually apparent change in appearance, e.g., by changing color from white to blue when the sensor is saturated. Such excessive fluid could cause device 15 to clog and thereafter perform unreliably. In one preferred form of the invention, colorimetric humidity/fluid sensor 35 is disposed in a proximal chamber 40 located between inlet 20 and the individual colorimetric enteric gas sensors 25A, 25B, 25C, 25D, 25E, etc. so that the colorimetric humidity/fluid sensor is readily visible to the surgeon and operating room personnel. In addition, proximal chamber 40 may also include a desiccant (e.g., silica granules) for quickly and consistently removing water vapor from the intraabdominal gas prior to the intraabdominal gas entering the individual colorimetric enteric gas sensors 25A, 25B, 25C, 25D, 25E, etc. It should be appreciated that in one preferred form of the invention, colorimetric humidity/fluid sensor 35 also performs as a dessicant, e.g., colorimetric humidity/fluid sensor 35 may comprise silica granules which act as a dessicant and change color from white to blue when saturated. In one preferred form of the invention, proximal chamber 40 comprises a single open chamber that communicates equally with each of the individual colorimetric enteric gas sensors 25A, 25B, 25C, 25D, 25E, etc. so as to ensure equal gas flow and pressurization of each of the individual colorimetric enteric gas sensors 25A, 25B, 25C, 25D, 25E, etc., whereby to facilitate detection accuracy.

In one preferred form of the invention, device 15 also comprises a plurality of rapid and reversible colorimetric CO₂ sensors 45A, 45B, 45C, 45D, 45E, etc. disposed within the interior of the cartridge for functioning as a control to confirm that the device is receiving gas flow from the intraabdominal cavity. More particularly, the outflow side of each of the individual colorimetric enteric gas sensors 25A, 25B, 25C, 25D, 25E, etc. are connected to an associated rapid and reversible colorimetric CO₂ sensor 45A, 45B, 45C, 45D, 45E, etc., wherein the rapid and reversible colorimetric CO₂ sensors are adapted to detect the presence of CO₂ in the gas stream received from the individual colorimetric enteric gas sensors 25A, 25B, 25C, 25D, 25E, etc. and indicate that presence by a visually-apparent change in appearance. The rapid and reversible colorimetric CO₂ sensors are disposed in device 15 so that the rapid and reversible colorimetric CO₂ sensor is readily visible to the surgeon and operating room personnel. In this way, the rapid and reversible colorimetric CO₂ sensors 45A, 45B, 45C, 45D, 45E, etc. can monitor and verify, in real-time, the patency of each individual colorimetric enteric gas sensor 25A, 25B, 25C, 25D, 25E, etc.

Preferably, device 15 has seals over its inlet 20 and outlets 30A, 30B, 30C, 30D, 30E, etc. which are broken upon use or, alternatively, has check valves (not shown) at its inlet 20 and outlets 30A, 30B, 30C, 30D, 30E, etc. so as to prevent ambient air/humidity from entering device 15 and causing failure/malfunction prior to usage and for preservation during long-term pre-operative storage.

Inlet 20 of device 15 has a connection (e.g., a luer lock connection) that allows device 15 to be quickly and securely connected to a passageway leading to the intraabdominal cavity, e.g., to the side gas valve of an access port such as access port 5B, 5C.

By allowing a small, constant amount of air from the intraabdominal cavity to vent through device 15 during the minimally invasive procedure or robotic procedure (or other procedure), device 15 can be used to monitor the gases present in the intraabdominal cavity, whereby to sense the presence of selected enteric gases that would indicate the possibility of a perforation of the bowel (and/or to sense for the concentrations of selected enteric gases that would indicate the possibility of a perforation of the bowel). Detection is indicated by a colorimetric change to one (or several) of the individual colorimetric enteric gas sensors 25A, 25B, 25C, 25D, 25E, etc. as they detect the presence of specific enteric gases and/or enteric gas concentrations.

As the abdomen is typically continually insufflated with approximately 15 PSI of positive pressure using CO₂, opening a side vent on access port 5B, 5C allows active and pressurized continual intraabdominal airflow through device 15. An important aspect of the present invention is that device 15 (including its constituent components inlet 20, colorimetric humidity/fluid sensor 35, individual colorimetric enteric gas sensors 25A, 25B, 25C, 25D, 25E, etc., the rapid and reversible colorimetric CO₂ sensors 45A, 45B, 45C, 45D, 45E, etc. and outlets 30A, 30B, 30C, 30D, 30E, etc.) are designed to operate at the insufflation pressure used for the surgical procedure, thus eliminating the need for suction pumps, etc.

More particularly, device 15 contains a plurality of separate individual colorimetric enteric gas sensors 25A, 25B, 25C, 25D, 25E, etc. (e.g., in the form of separate individual detector tubes), each of which is specifically designed to change color in the presence of a particular gas found only in the bowel, e.g., hydrogen sulfide, dimethylsulfide, methanethiol (aka methyl mercaptan), and can be extremely sensitive (e.g., down to 0.25 parts per million for some compounds). Additionally and/or alternatively, device 15 contains separate individual colorimetric enteric gas sensors 25A, 25B, 25C, 25D, 25E, etc. (preferably in the form of separate individual detector tubes), each of which is specifically designed to change color in the presence of a threshold concentration of a particular gas found only in the bowel, e.g., a threshold concentration of methane, hydrogen gas, etc. The separate individual colorimetric gas sensors 25A, 25B, 25C, 25D, 25E, etc. are provided in parallel, and are individually labeled, so that the presence and/or concentration of the enteric gas detected by each individual colorimetric gas sensor 25A, 25B, 25C, 25D, 25E, etc. can be instantaneously seen and known through color change.

For purposes of the present invention, the terms “colorimetric” and/or “colorimetric sensor” and the like (e.g., the individual colorimetric enteric gas sensors 25A, 25B, 25C, 25D, 25E, etc., the colorimetric humidity/fluid saturation sensor 35, the rapid and reversible colorimetric CO₂ sensors 45A, 45B, 45C, 45D, 45E, etc.) are intended to mean an element capable of detecting the presence of a target substance and/or a threshold concentration of a target substance and indicating that presence by a visually apparent change in appearance, i.e., a “change in color”, a “color change”, etc.

For purposes of the present invention, the terms “change in color” and/or “color change” and the like are intended to refer to a visually detectable change in one or more of hue, saturation and brightness, including intensity and opacity, and distance of color change down the length of each individual colorimetric gas sensor 25A, 25B, 25C, 25D, 25E, etc.

In one preferred form of the invention, the individual colorimetric enteric gas sensors 25A, 25B, 25C, 25D, 25E, etc. comprise detector tubes each containing a substance for reacting with its target enteric gas (e.g., hydrogen sulfide, dimethylsulfide, methanethiol aka methyl mercaptan, etc.) and/or a threshold concentration of a target enteric gas (e.g., methane, hydrogen, etc.) and providing a visually-detectable color change for indicating the presence of the target enteric gas and/or threshold concentration of a target enteric gas.

In one preferred form of the invention, the individual colorimetric enteric gas sensors 25A, 25B, 25C, 25D, 25E, etc. comprise detector tubes sold by Dräger of Andover, Mass., USA, e.g., Dräger detector tube 8101831 for detecting hydrogen sulfide; Dräger detector tube 6728451 for detecting dimethylsulfide; Dräger detector tube 6728981 for detecting methanethiol aka methyl mercaptan; Dräger detector tube CH20001 for detecting methane; Dräger detector tube 8101511 for detecting hydrogen, etc.

Preferably device 15 is constructed out of small, low-cost, and “environmentally-friendly” components, so that the device may be easily disposed of after use.

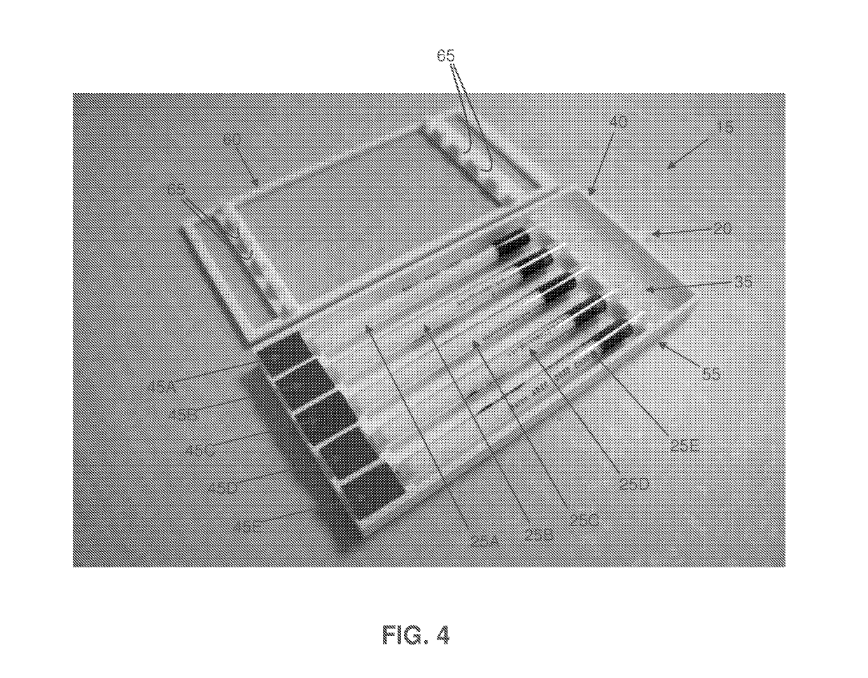

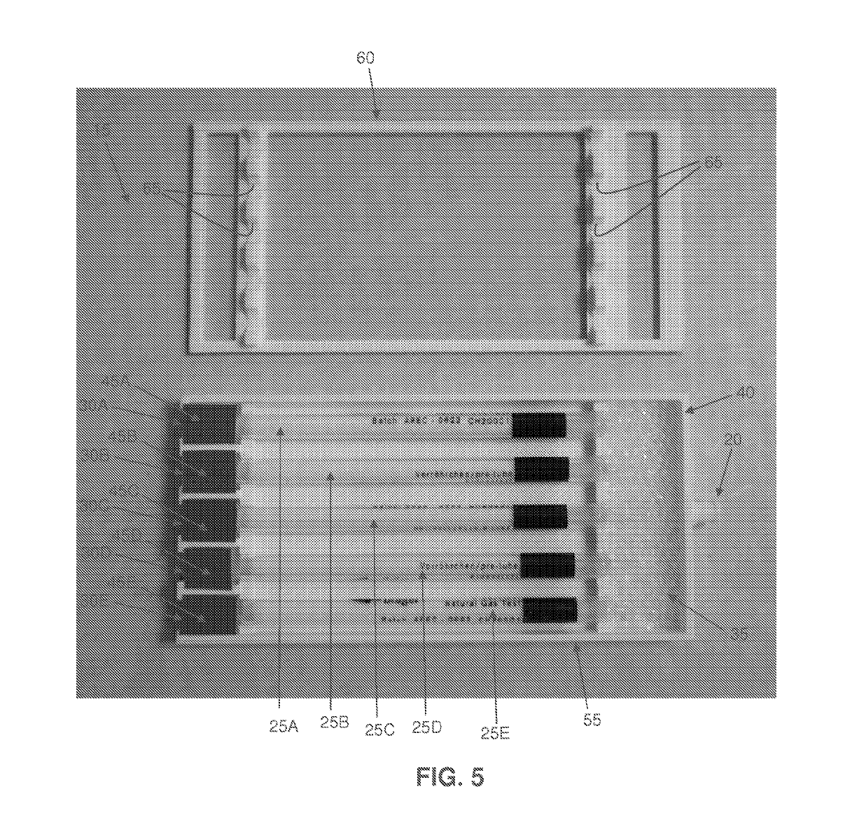

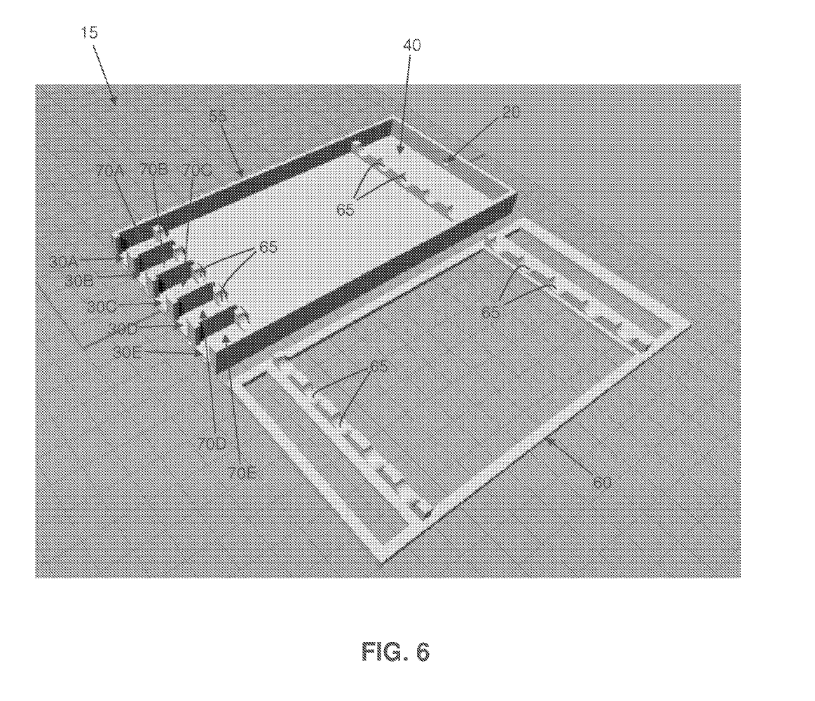

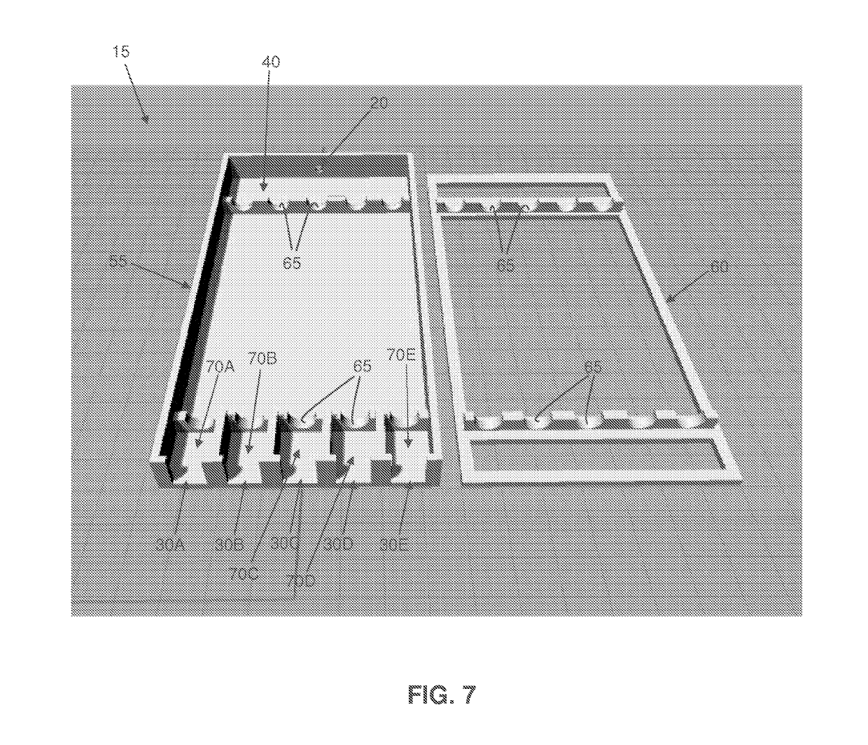

In one preferred form of the invention, device 15 comprises a housing 50 having two halves 55, 60 which are united during manufacturing so as to together form the complete housing for device 15. See FIGS. 4-7. These two halves 55, 60 assemble together during manufacturing so as to form a hollow structure with appropriate cavities and chambers for receiving the individual colorimetric enteric gas sensors 25A, 25B, 25C, 25D, 25E, etc., the colorimetric humidity/fluid saturation sensor 35 and the rapid and reversible colorimetric CO₂ sensors 45A, 45B, 45C, 45D, 45E, etc. By way of example but not limitation, the two halves 55, 60 assemble together so as to provide inlet 20, proximal chamber 40 which is in fluid communication with inlet 20 (and which receives colorimetric humidity/fluid saturation sensor 35 and preferably a dessicant as well, or a colorimetric humidity/fluid saturation sensor 35 which also acts as a dessicant), tube seats 65A, 65B, 65C, 65D, 65E, etc. which receive the individual colorimetric enteric gas sensors 25A, 25B, 25C, 25D, 25E, etc. so that the individual colorimetric enteric gas sensors are each supported in fluid communication with proximal chamber 40, chambers 70A, 70B, 70C, 70D, 70E, etc. which are in fluid communication with the individual colorimetric enteric gas sensors 25A, 25B, 25C, 25D, 25E, etc. (and which receive the rapid and reversible colorimetric CO₂ sensors 45A, 45B, 45C, 45D, 45E, etc., respectively), and outlets 30A, 30B, 30C, 30D, 30E, etc. which are in fluid communication with the rapid and reversible colorimetric CO₂ sensors 45A, 45B, 45C, 45D, 45E, etc., respectively, which are contained in chambers 70A, 70B, 70C, 70D, 70E, etc., respectively.

In one preferred form of the invention, device 15 is initially connected to the first access port 5 which is inserted into the abdomen (which is traditionally the access port with the highest risk of an unplanned enterotomy) to see if there is, immediately, the presence of a gas or gas concentration indicative of bowel perforation. Device 15 may then remain in place for the duration of the procedure so as to serve as a “sentinel” to detect the occurrence of an unplanned enterotomy. At the end of the procedure (as well as intermittently during the procedure), device 15 may be checked for any color change that would be indicative of a bowel perforation.

Preferably device 15 also includes the aforementioned colorimetric humidity/fluid saturation sensor 35 that changes color upon fluid saturation (and preferably a dessicant as well), whereby to monitor how much liquid/water vapor has entered device 15, since excess fluid content could cause device 15 to clog and thereafter perform unreliably. In one preferred form of the invention, colorimetric humidity/fluid sensor 35 also performs as a dessicant, e.g., colorimetric humidity/fluid sensor 35 may comprise silica granules which act as a dessicant and change color from white to blue when saturated.

And device 15 preferably comprises the aforementioned plurality of rapid and reversible colorimetric CO₂ sensors 45A, 45B, 45C, 45D, 45E, etc. for functioning as a control to confirm that the device is receiving gas flow from the intraabdominal cavity and that the gas flow is passing through each individual colorimetric enteric gas sensor 25A, 25B, 25C, 25D, 25E, etc. In one preferred form of the invention, the rapid and reversible colorimetric CO₂ sensors 45A, 45B, 45C, 45D, 45E, etc. may comprise the Nellcor Easy Cap II CO₂ detector available from Covidien PLC of Dublin, Ireland.

In the foregoing disclosure, device 15 is discussed in the context of detecting naturally occurring enteric gases (e.g., hydrogen sulfide, dimethylsulfide, methanethiol, aka methyl mercaptan, and/or threshold concentrations of methane, hydrogen, etc.). However, if desired, device 15 can be configured to sense for the presence and/or concentrations of non-naturally-occurring enteric gases, e.g., a gas whose production is induced by the digestion of a selected substance, and/or a gas which is deliberately introduced into the intestinal tract upstream or downstream of the surgical site.

Thus, for the purposes of the present invention, the term “enteric gas” is intended to refer to substantially any gas which may be located within the intestinal tract, e.g., a naturally-occurring gas (such as hydrogen sulfide, dimethylsulfide, methanethiol, aka methyl mercaptan, and/or threshold concentrations of methane, hydrogen, etc.), a non-naturally-occurring gas (such as a gas whose production is induced by the digestion of a selected substance, a gas which is deliberately introduced into the intestinal tract upstream or downstream of a surgical site, etc.), etc., and the term “enteric gas sensor” is intended to refer to a sensor which may be used to detect an enteric gas.

In the foregoing disclosure, colorimetric enteric gas sensors 25A, 25B, 25C, 25D, 25E, etc. are shown as being disposed in parallel within device 15. However, if desired, colorimetric enteric gas sensors 25A, 25B, 25C, 25D, 25E, etc. could be disposed serially within device 15.

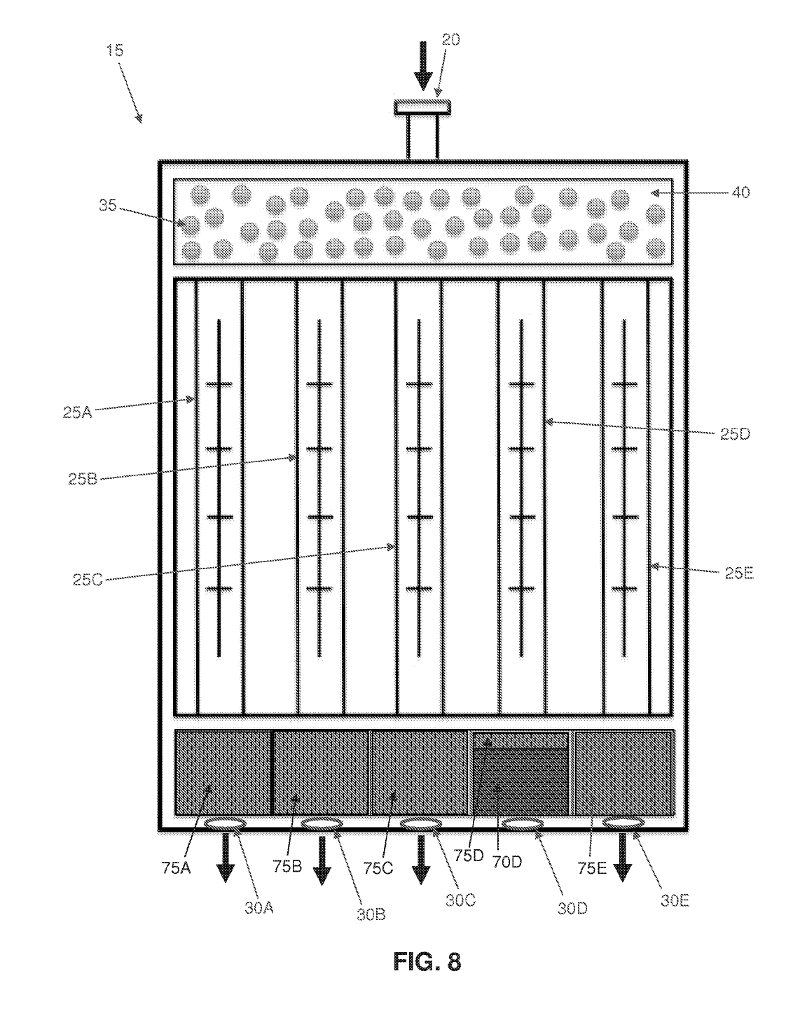

In the foregoing disclosure, rapid and reversible colorimetric CO₂ sensors 45A, 45B, 45C, 45D, 45E, etc. are provided to monitor and verify, in real-time, the patency of each individual colorimetric enteric gas sensor 25A, 25B, 25C, 25D, 25E, etc. However, if desired, alternative means may be provided for monitoring and verifying, in real-time, the patency of each individual colorimetric enteric gas sensor 25A, 25B, 25C, 25D, 25E, etc. By way of example but not limitation, and looking now at FIG. 8, a plurality of flaps 75A, 75B, 75C, 75D, 75E, etc. may be hingedly mounted within each of the chambers 70A, 70B, 70C, 70D, 70E, etc. such that when gas is flowing through each individual colorimetric enteric gas sensor 25A, 25B, 25C, 25D, 25E, etc., the corresponding flaps 75A, 75B, 75C, 75D, 75E, etc. are “pushed upward” so that they cover the top of the corresponding chamber 70A, 70B, 70C, 70D, 70E, etc. and are thereby visible to the surgeon and operating room personnel; however, when gas is not flowing through an individual colorimetric enteric gas sensor 25A, 25B, 25C, 25D, 25E, etc., the corresponding flap 75A, 75B, 75C, 75D, 75E, etc. is not “pushed upward” so that it covers the top of the corresponding chamber 70A, 70B, 70C, 70D, 70E, etc. and hence are not visible to the surgeon and operating room personnel. By providing appropriate visual differentiation between the tops of the flaps 75A, 75B, 75C, 75D, 75E, etc. and the interior surfaces of the chambers 70A, 70B, 70C, 70D, 70E, etc. (e.g., different colors, different patterns, different text, etc.), the patency of each individual colorimetric enteric gas sensor 25A, 25B, 25C, 25D, 25E, etc. can be visually indicated to the surgeon and operating room personnel.

It should be appreciated that the present invention may be used to evaluate and assess the integrity and/or patency of any hollow organ repair, anastomosis or procedure relating to the wall of a hollow organ, performed in either a minimally invasive procedure, and/or a robotic procedure (and/or other procedure), including an open surgical procedure. Any leakage of detectable gases (either native or introduced) may be detected by providing a device 15 fitted with an appropriate set of individual colorimetric gas sensors 25A, 25B, 25C, 25D, 25E, etc.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. An in-line enterotomy detection device for use in a surgical procedure wherein access to an internal operating field is provided through a plurality of access ports and further wherein a pressurized insufflating gas is delivered to the internal operating field through one access port and vented from the internal operating field through another access port, said in-line enterotomy detection device comprising:
a housing having an inlet, an outlet, and a passageway connecting said inlet to said outlet, said inlet being configured for connection to an access port venting gas from the internal operating field and said outlet being configured to vent gas from said in-line enterotomy detection device; and
at least one non-electrical colorimetric enteric gas sensor disposed within said passageway for determining at least one of the presence of a selected enteric gas and the concentration of a selected enteric gas and indicating that presence by a visually-apparent change in appearance.

2. An in-line enterotomy detection device according to claim 1 wherein said in-line enterotomy detection device is operative at the insufflation pressure used for the surgical procedure without requiring supplemental pressurization or suction.

3. An in-line enterotomy detection device according to claim 1 wherein said at least one non-electrical colorimetric enteric gas sensor comprises at least one tube having a first opening in fluid communication with said inlet, a second opening in fluid communication with said outlet, and a reactive material for providing a visually-apparent change in appearance when exposed to at least one of a selected enteric gas and a concentration of a selected enteric gas.

4. An in-line enterotomy detection device according to claim 3 wherein said at least one non-electrical colorimetric enteric gas sensor is adapted to provide a visually-apparent change in appearance when exposed to a selected enteric gas.

5. An in-line enterotomy detection device according to claim 3 wherein said selected enteric gas comprises a naturally-occurring enteric gas.

6. An in-line enterotomy detection device according to claim 5 wherein said selected enteric gas comprises hydrogen sulfide.

7. An in-line enterotomy detection device according to claim 5 wherein said selected enteric gas comprises dimethylsulfide.

8. An in-line enterotomy detection device according to claim 5 wherein said selected enteric gas comprises methanethiol, aka methyl mercaptan.

9. An in-line enterotomy detection device according to claim 3 wherein said selected enteric gas comprises a non-naturally-occurring enteric gas.

10. An in-line enterotomy detection device according to claim 9 wherein said non-naturally-occurring enteric gas comprises a gas whose production is induced by the digestion of a selected substance.

11. An in-line enterotomy detection device according to claim 9 wherein said non-naturally-occurring enteric gas comprises a gas which is deliberately introduced into the intestinal tract upstream or downstream of the surgical site.

12. An in-line enterotomy detection device according to claim 3 wherein said at least one non-electrical colorimetric enteric gas sensor is adapted to provide a visually-apparent change in appearance when exposed to a concentration of a selected enteric gas.

13. An in-line enterotomy detection device according to claim 12 wherein said selected enteric gas comprises methane.

14. An in-line enterotomy detection device according to claim 12 wherein said selected enteric gas comprises hydrogen.

15. An in-line enterotomy detection device according to claim 12 wherein said at least one non-electrical colorimetric enteric gas sensor has a length, and further wherein said visually-apparent change in appearance occurs along a length of said at least one non-electrical colorimetric enteric gas sensor in proportion to the concentration of said selected enteric gas.

16. An in-line enterotomy detection device according to claim 1 further comprising a colorimetric humidity/fluid sensor disposed within said passageway between said inlet and said at least one non-electrical colorimetric enteric gas sensor for detecting if excessive fluid is present in the gas received in said inlet and indicating that presence by a visually-apparent change in appearance.

17. An in-line enterotomy detection device according to claim 16 wherein said colorimetric humidity/fluid sensor comprises a dessicant.

18. An in-line enterotomy detection device according to claim 16 wherein said colorimetric humidity/fluid sensor is a dessicant.

19. An in-line enterotomy detection device according to claim 18 wherein said colorimetric humidity/fluid sensor comprises silica granules.

20. An in-line enterotomy detection device according to claim 1 further comprising a patency indicator disposed within said passageway between said at least one non-electrical colorimetric enteric gas sensor and said outlet for monitoring and verifying, in real-time, the patency of said at least one non-electrical colorimetric enteric gas sensor.

21. An in-line enterotomy detection device according to claim 20 wherein said patency indicator comprises a rapid and reversible colorimetric CO₂ sensor disposed within said passageway between said at least one non-electrical colorimetric enteric gas sensor and said outlet for detecting the presence of CO₂ in the gas received in said inlet and indicating that presence by a visually-apparent change in appearance.

22. An in-line enterotomy detection device according to claim 20 wherein said patency indicator comprises a visually-apparent flap disposed within said passageway between said at least one non-electrical colorimetric enteric gas sensor and said outlet.

23. An in-line enterotomy detection device according to claim 1 wherein said at least one non-electrical colorimetric enteric gas sensor comprises a plurality of non-electrical colorimetric enteric gas sensors.

24. An in-line enterotomy detection device according to claim 23 wherein said plurality of non-electrical colorimetric enteric gas sensors are disposed in parallel.

25. An in-line enterotomy detection device according to claim 24 further comprising a plurality of rapid and reversible colorimetric CO₂ sensors disposed within said passageway between said plurality of non-electrical colorimetric enteric gas sensors and said outlet for detecting the presence of CO₂ in the gas received in said inlet and indicating that presence by a visually-apparent change in appearance.

26. An in-line enterotomy detection device according to claim 25 wherein one rapid and reversible colorimetric CO₂ sensor is disposed within said passageway between each of said plurality of non-electrical colorimetric enteric gas sensors and said outlet.

27. An in-line enterotomy detection device according to claim 26 wherein said outlet comprises a plurality of outlets, and further wherein one outlet is associated with each rapid and reversible colorimetric CO₂ sensor.

28. An in-line enterotomy detection device according to claim 23 wherein said plurality of non-electrical colorimetric enteric gas sensors are disposed in series.

29. An in-line enterotomy detection device according to claim 1 wherein said inlet comprises a portion of a luer lock connection.

30. A novel method for detecting perforation of the bowel in a surgical procedure wherein access to an internal operating field is provided through a plurality of access ports and further wherein a pressurized insufflating gas is delivered to the internal operating field through one access port and vented from the internal operating field through another access port, said method comprising:
providing an in-line enterotomy detection device comprising:
a housing having an inlet, an outlet, and a passageway connecting said inlet to said outlet; and
at least one non-electrical colorimetric enteric gas sensor disposed within said passageway for determining at least one of the presence of a selected enteric gas and the concentration of a selected enteric gas and indicating that presence by a visually-apparent change in appearance;
connecting said inlet to an access port venting gas from the internal operating field; and
observing the appearance of said at least one non-electrical colorimetric enteric gas sensor during the surgical procedure.
